(19) United States
(12) Patent Application Publication
(10) Pub. No.: US 2012/0107919 A1
(43) Pub. Date: May 3, 2012

(54) Method for Producing Biomass and Photobioreactor for Cultivating Phototrophic or Mixotrophic Organisms or Cells

(75) Inventors: Juergen Broneske, Nuthe-Urstromtal (DE); Otto Pulz, Nuthetal (DE); Thomas Rothe, Nuthetal (DE); Karsten Schmidt, Wandlitz (DE); Rainer Weidner, Koenigs Wusterhausen (DE)

(73) Assignee: IGV INSTITUT FUER GETREIDEVERARBEITUNG GMBH, Bergholz-Rehbruecke (DE)

(21) Appl. No.: 13/380,918

(22) PCT Filed: Jun. 22, 2010
(86) PCT No.: PCT/DE10/50039

§ 371 Date: Dec. 27, 2011

(30) Foreign Application Priority Data
Jun. 24, 2009 (DE) 102009027175.9

(51) Int. Cl. C12N 1/12; C12M 1/42

(52) U.S. Cl. 435/257.1; 435/292.1

(57) ABSTRACT

According to the proposed method for producing biomass, the organisms or cells in a suspension kept in circulation in a photobioreactor are cultivated with introduction of light and of at least CO₂ as a nutrient. For the cultivation, the suspension is introduced via at least one introduction organ in an upper region of a culturing space and its downward movement is slowed down by at least one inner element that has a horizontally extending grid, screen or net structure and that is disposed in the culturing space. The suspension is converted into a plurality of drops on this structure. The drops pass through a drop cycle, by means of which the downward movement of the suspension is slowed down and a particularly intensive exposure of the organisms or cells contained in the nutrient solution to nutrients and light introduced into the reactor is assured.

The invention relates to a method for producing biomass composed of phototrophic or mixotrophic organisms or cells. Without being limited thereto, the invention particularly refers to the production of biomass from lower plants, such as microalgae or mosses. Further, the invention relates to a photobioreactor that can be used for conducting the method for cultivating phototrophic or mixotrophic organisms or cells.

The production of biomass composed of phototrophic and mixotrophic organisms or cells, in particular of microalgae, has been increasingly gaining importance. The biomass produced in this way has been utilized meanwhile for the most varied of purposes. For example, it serves for the production of nutritional physiologically high-quality food and dietary supplements, as an additive for dermatological medications or cosmetic products, or also for the production of energy sources. Central components of corresponding equipment for biomass production are bioreactors for cultivating organisms or cells. While corresponding bioreactors in the past have been designed mostly on the laboratory scale and thus with relatively small production capacity, the installation of large-scale production equipment is now being promoted.

With respect to the production of biomass based on phototrophic or mixotrophic organisms or cells, the problem exists of providing photobioreactors that, on the one hand, provide a large volume for cultivating organisms or cells, and in which, on the other hand, despite the large quantities of biomass arising during cultivation within the reactor, there is assured a sufficient and uniform provision of the organisms or cells to be cultivated with the nutrients and with light necessary for this purpose.

One known possibility for biomass production is the use of tube bioreactors, in which a suspension containing organisms or cells is conducted through glass tubes disposed in a light-filled reactor space. In fact, good cultivation conditions are rather well provided by means of suitable glass tube reactors with respect to providing organisms with nutrients and light, but such reactors are rather unsuitable for mass production of biomass, particularly from the viewpoint of cost.

Another concept consists of introducing a suspension containing organisms or cells conducted in a circuit in the upper region of a culturing space of the photobioreactor via suitable introduction organs (for example, spray nozzles) and after this, to slow down the downward movement of the suspension produced by gravitational effect by the arrangement of suitable inner elements or fittings in the culturing space, so that an intensive exposure of the organisms or cells to the nutrients and light introduced into the reactor is assured. According to known solutions, the above-named inner elements involve, for example, a plurality of sheets of fabric extending vertically and disposed parallel to one another, so-called matrices or sheets. The organisms or cells of the suspension introduced above these fabric sheets are at least partially immobilized on the fabric sheets, which are usually composed of a hydrophilic material. In this way, they are intensively subjected for a longer time to the light and the nutrients produced directly in the reactor or guided to it. Of course, with increasing accumulation of biomass on the fabric sheets, the flow of light through the reactor or its culturing space is increasingly adversely affected. In particular, the material sheets that are not disposed in the outer regions become increasingly turbid, so that optimal cultivating conditions no longer exist for the biomass deposited on them. In addition, in bioreactors of this design, in which the organisms or cells are immobilized in the way described above to appropriate material sheets, the harvesting of the biomass is relatively complicated after the cultivating process has terminated. The corresponding biomass is rinsed from the material sheets, for example, by means of high-pressure cleaners. This procedure is time-consuming and in general requires the use of human labor in the harvesting.

The object of the invention is to provide an alternative solution for large-scale production of biomass, which is designed in such a way that it particularly makes possible the provision of favorable cultivating conditions for the cultivation of very large quantities of biomass and still permits a comparatively simple construction of the corresponding production equipment. A method will be described and a photobioreactor for cultivating phototrophic or mixotrophic organisms or cells will be provided for this purpose.

The object is achieved by a method with the features of the principal claim. A photobioreactor achieving the object is characterized by the first independent device claim. Advantageous embodiments or enhancements of the invention are given by the respective subclaims.

In contrast to autotrophic organisms, which are nourished from inorganic substances, phototrophic organisms or cells are nourished and multiplied under action of energy, particularly light, and by assimilation of carbon dioxide, in particular. Corresponding to their name, mixotrophic organisms represent a mixed form, which, in addition to carbon dioxide, can also assimilate organic substances and thus generally drive photosynthesis. The method accordingly relates to the production of biomass composed of organisms or cells, whose growth and propagation in any case takes place by way of photosynthesis and with provision of nutrients such as carbon dioxide, in particular. The method preferably serves for producing biomass composed of phototrophic or mixotrophic microalgae, however, as has already been mentioned initially, but not being limited thereto.

According to the proposed method for producing biomass composed of phototropic or mixotrophic organisms or cells, the organisms or cells are cultivated in a suspension kept in circulation in a photobioreactor. For the cultivation, during the circulation of the suspension, energy in the form of natural or artificial light and at least CO₂ as a gaseous nutrient are conducted to the organisms or cells. For this purpose, the suspension is introduced by at least one introduction organ in an upper region of a culturing space of a photobioreactor. The downward movement of the suspension introduced in the culturing space, which is executed by the effect of gravity, is slowed down by at least one suitable inner element disposed in the culturing space, and thus an intensive exposure of the organisms or cells to the light entering into the culturing space or produced therein and to the gaseous nutrient(s) is assured. The suspension collecting at the bottom of the culturing space is finally conducted repeatedly to the at least one introduction organ for providing the circulation by means of a pumping system. After terminating the cultivation of the organisms or cells, the biomass is harvested by separating it from the suspension. This is carried out with the aid of appropriate means for separation, such as separators, filters or sieving devices.

According to the invention, the suspension for the mentioned slowing down of the downward movement in the culturing space is converted to a plurality of drops. In this way, the suspension is conducted to a structure of one or more inner elements of the culturing space designed for this purpose for the formation of the drops. For the parts of the suspension converted into drops according to the invention, each individual drop passes through at least once a drop cycle executed as follows during passage through the culturing space:
formation of the drop on the structure provided for this in the culturing space,
gradual enlargement of the drop up to a maximum size, during which the organisms or cells contained in the drop are exposed to light and to the one or more gaseous nutrients and are multiplied.
after reaching a maximum size, dripping the drop down into a collection region at the bottom of the culturing space or onto another structure of an inner element of the culturing space that produces drops.

By converting the suspension into a plurality of drops, many small surfaces are created, each of which forms an interface between liquid (the suspension containing organisms or cells) and gas (the atmosphere of the inside space of the reactor with gaseous nutrients contained therein), which in total form a very large surface or interface, on which the organisms or cells are exposed in a particularly intensive manner to light and nutrients. Due to the small-volume drops, the average path length of the organisms or cells to the interface is also minimized. During the enlargement phase of the drop, the latter can assume different forms, one possible form being the droplet form which is typical in a fluid-dynamic sense and which is approximately round on the bottom and tapers into a point on top. Which form the drop assumes each time thus particularly depends on the nature of the suspension, on the one hand, and on the configuration of the inner elements bringing about the drop formation, on the other hand. However, to a certain extent, the form can also be controlled by how the suspension is introduced via the at least one introduction organ. As will be discussed below, specific drop forms are preferably to be targeted.

It has also been shown that due to the light effect and the heating that accompanies it as well as material transport caused by differences in concentration, turbulence arises, so that the organisms or cells repeatedly linger at the interfaces, whereby a particularly strong exposure to light and nutrients occurs. Thus far, good cultivation results have been established by experiments. From these, it has therefore been found that the slowing down of the downward movement of the suspension in the culturing space, which is advantageously achieved without immobilizing the biomass, and the dynamic processes occurring in the drops advantageously influence the cultivation result.

Relative to the already mentioned drop form, a lens-shaped drop form has been demonstrated to be very advantageous, since in this way an improved or very concentrated light incidence in the drop and thus a very good provision of light energy to the organisms or cells is achieved, particularly under low-light conditions. According to an advantageous embodiment of the method, the suspension is thus passed through an appropriate formation of the inner element and/or an appropriate control of its introduction via the at least one introduction organ for the formation of predominantly lens-shaped drops.

The generation of mist-like drops, i.e., spherical drops in the microrange, however, has also been demonstrated to be advantageous. An optimal interaction between organisms or cells and the photons in the entire culturing space or cultivating space is produced in this way.

According to one possible method embodiment, for harvesting the biomass, the drops formed on the structures of the culturing space, which are provided for this purpose, are removed from these structures by the effect of vibration or by shock-like vibrations. The suspension with the organisms or cells contained therein, which collects at the bottom of the culturing space, as has already been mentioned, is then conducted to means for separating the organisms or cells from the suspension. According to a possible variant of this method embodiment, the drops are removed from the structures of the culturing space by means of ultrasound acting on the structures.

According to another method embodiment, the drops are removed from the structures of the culturing space by blowing off the drops by means of a fan. The suspension collecting on the bottom is then also again conducted to means for separating the organisms or cells from the suspension. Finally, there is another possibility for harvesting the biomass that has been produced by rinsing off the drops of the suspension, which have been formed on the structures of the culturing space provided therefor, by means of a rinsing fluid. In this case, instead of the suspension, the appropriate rinsing fluid is introduced by means of the at least one introduction organ in the culturing space. As a consequence, only a reversing of the conducting paths of the existing pumping system is necessary for this.

Independently from the procedure selected for removing the drops from the structures of the culturing space, it is provided according to an advantageous embodiment of the method, to remove films of the suspension that absorb the organisms or cells and that remain on the inner elements of the culturing space by post-rinsing with a rinsing fluid. For example, water or a nutrient solution is used as the rinsing fluid for both the possible removal of the drops from the structures of the inner elements conducted by means of a rinsing fluid as well as for the optional post-rinsing for the purpose of removing the remaining suspension films.

The photobioreactor that achieves the object and that can be used for the cultivation when conducting the method is composed of
a culturing space, which is permeated by natural light or artificial light produced from outside or inside the culturing space, and in which at least CO₂ is introduced as a gaseous nutrient,
at least one introduction organ, by means of which the organisms or cells contained in the suspension are introduced in an upper region of the culturing space for exposure to light and to the one or more nutrients,
at least one inner element disposed in the culturing space, by means of which the downward movement of the suspension in the culturing space, which occurs due to the effect of gravity, is delayed, as well as
a pumping system, by means of which the suspension collecting at the bottom of the culturing space is conducted repeatedly to the at least one introduction organ for providing circulation.

According to the invention, at least one inner element having a grid, screen, or net structure extending horizontally is disposed in the culturing space of the photobioreactor underneath the at least one introduction organ. The suspension containing the organisms or cells, which is introduced into the culturing space, is conducted through the above-named structure according to the invention for the formation of a plurality of drops, which, after they have formed, increase in size and in each case, after reaching a maximum drop size, are dripped down into the collecting region at the bottom of the culturing space or onto another structure of an inner element of comparable type that produces drops and is disposed in the culturing space underneath the previously named structure.

The structure according to the invention that is horizontally disposed in the culturing space can be created with use of different materials and can be configured in various ways with respect to its geometry. Accordingly, for example, the use of flexible materials involves a rather net-like structure, whereas grid or screen structures are provided by means of solid materials, whereby the latter differ only slightly with respect to their geometric formation, so that a corresponding structure can be optionally called a grid structure or a screen structure. In each, case, however, all named structures (grid, screen or net structures) have the same effect or are designed for achieving the same goal, namely for converting the suspension entering into the culturing space into a plurality of drops. In the following, therefore, for simplicity and generalization, a grid structure shall be described, whereby the corresponding representations refer equally to net or screen structures.

In each case, depending on the nature of the suspension and the grid geometry, i.e., the distances between the grid crosspieces and grid nodes, the drops produced according to the invention develop either on a grid node, a grid crosspiece, or also, by bridging the grid crosspieces to form a boundary around them, in a grid window. The deciding factor, however, is thus only that the vertical movement of the suspension containing the organisms or the cells is slowed down timewise in the culturing space due to the formation of drops, and in this case, the drop cycle, which is explained in the statements relative to the method and which promotes the exposure of the organisms or cells to light and nutrients, is executed. The at least one grid structure or grid-like structure (screen or net structure) according to a preferred embodiment of the photobioreactor according to the invention is composed of a hydrophobic material. Thus, it is assumed that the use of hydrophobic materials is advantageous as long as no immobilizing processes are basically executed thereby. Also, the parts of the suspension remaining on the reactor walls or the walls of the culturing space and the inner elements as a film should be fewer in this case, so that the expenditure for possible post-rinsing after the cultivation is reduced. The material should also be preferably selected so that hydraulically smooth surfaces are formed on the grid crosspieces or the net meshes or the intermediate spaces between the holes or windows in the screen. Film formation is also minimized thereby. In addition, the use of transparent materials for the grid structure can be viewed as advantageous, since in this case a uniform flooding of the culturing space with light is hindered the least, and a sufficient provision of light to the organisms or cells is an important prerequisite for cultivation success. Light-guiding materials are particularly advantageous for providing the grid structure. However, the use of white materials has also been demonstrated to be a practical success.

It should be mentioned here that the conversion of the introduced suspension into a plurality of drops, which is provided according to the method of the invention, can be optionally also conducted with similar structures on inner elements disposed vertically or inclined to the horizontal within a bioreactor. For example, structures or surface structures forming drops also appear basically conceivable in “Christmas-tree shape” on pyramidal elements or on inner elements. This explicitly leaves open the claimed method, so that it can be conducted optionally also independently of the protected solution for the configuration of a photobioreactor. Relative to the claimed device, however, as stated and claimed above, the invention refers to a photobioreactor that is viewed as practical, and, relative to the arrangement of the inner elements that slow down the downward directed movement of the suspension, it clearly differs from the prior art, where the corresponding inner elements in the culturing space are disposed horizontally. In this sense, inner elements that have slight inclinations that are particularly due to tolerances of their geometry and the means serving for their arrangement and fastening are also viewed as being disposed horizontally.

A preferred embodiment of the photobioreactor according to the invention is thus provided by disposing several grid, screen or net structures that extend horizontally in the culturing space in a cascade, one underneath the other. The grid window, the net meshes or the break-throughs of the individual grid structures or grid-like structures parallel to one another, and also the grid itself, can thus be of different size throughout as a function of the geometry of the culturing space and/or the nature of the suspension, whereby it particularly depends on the type of suspension as well as on the light conditions given each time and in the case of natural light, varying from one application site to another, as to whether the grid window or meshes or break-throughs in the vertical direction of movement of the suspension are large or small.

In an additionally provided embodiment of the invention, a dripping space is provided by disposing several nets, cords, strips or chains extending vertically downward in the culturing space, from the one horizontal grid structure present or—in the case of several parallelly disposed grid structures—from the last horizontal grid structure. The drops of suspension dripping down from the respective grid structure run downward through these nets, cords, strips or chains in the direction of the bottom of the culturing space. It has been shown here that the use of hydrophilic materials is advantageous for a dripping space provided by means of the above-named arrangements.

The photobioreactor according to the invention can still be enhanced by providing a unit for generation of shock-type vibrations in order to remove the drops remaining on the at least one grid structure after terminating the circulation of the suspension. In another, also advantageous embodiment, for harvesting the biomass, the photobioreactor has an ultrasonic transmitter, the ultrasonic vibrations of which act on the grid structure to remove the drops remaining on the respective grid structure after terminating the circulation of the suspension. In another possible embodiment, a fan is disposed in the photobioreactor or in its culturing space for removing the drops remaining on the grid structure after the cultivation and thus for supporting the harvesting process.

The invention will be explained in more detail below on the basis of drawings and embodiment examples, whereby the invention is represented in the following with reference to the cultivation of phototrophic microalgae as an example. The processes shown, however, are set up in the same or a basically comparable way for the cultivation of other phototrophic or mixotrophic organisms or cells. The following are shown in the appended drawings:

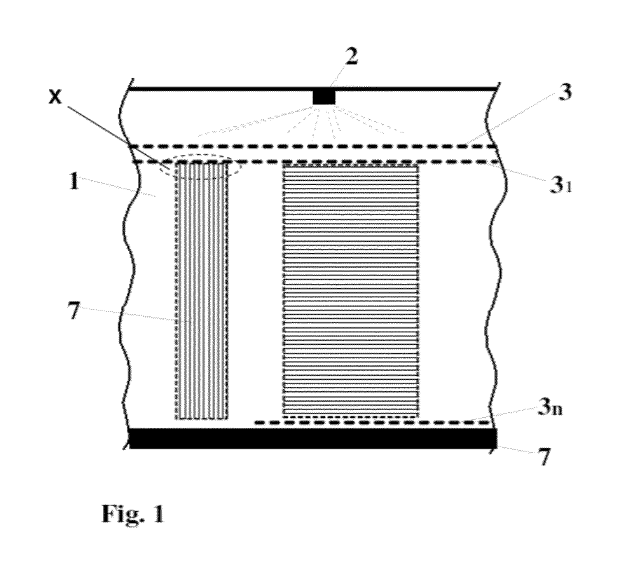

FIG. 1: the schematic representation of two possibilities for arranging the inner elements according to the invention in the culturing space of a photobioreactor,

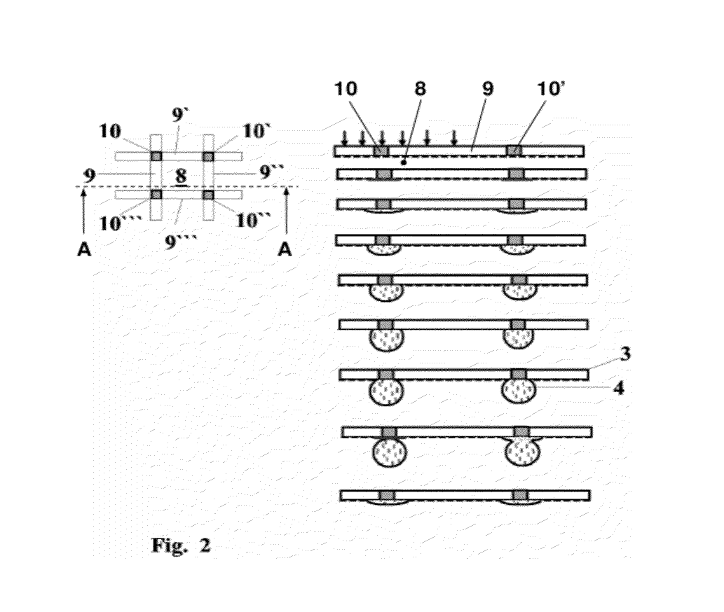

FIG. 2: the drop cycle set up according to the invention in a schematic representation,

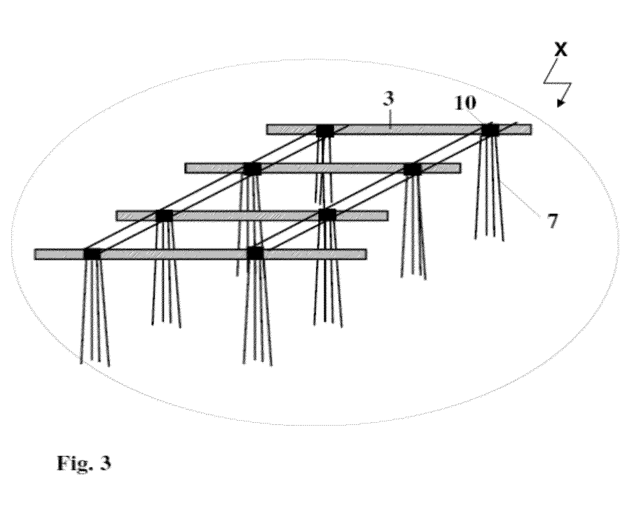

FIG. 3: the detail X of FIG. 1 in spatial representation,

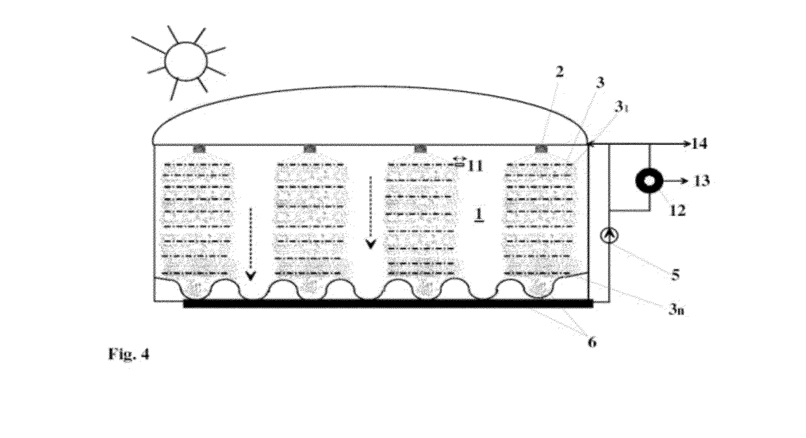

FIG. 4: the possible formation of the culturing space of a photobioreactor according to the invention in a schematic representation.

FIG. 1 shows two examples of possible configurations of a bioreactor according to the invention with inner elements 3, 3₁, 3ₙ,7 in a schematic representation. On the right side of the figure, a variant of the embodiment is shown, in which several inner elements 3, 3₁, 3ₙ with grid or screen structures are disposed horizontally, parallel to one another. In this arrangement, the parts of the suspension containing the microalgae, which has been converted to drop form, pass through the drop cycle several times, which is explained below on the basis of FIG. 2. In contrast, the left side relates to a possible embodiment variant, in which several strips 7 are disposed vertically on the bottom of an inner element 3₁ with a grid structure that converts the suspension into a plurality of drops 4. In this case, after dripping down from the grid structure of the inner element 3₁, drops 4 of the suspension run downward over the vertical structure, in this respect acting as a dripping space. In both variants shown in FIG. 1, the suspension is finally collected at the bottom or in a collecting region 6 of the culturing space 1 and again conducted to the introduction organ 2 disposed above the grid structure. The latter involves a spray nozzle, for example.

In FIG. 2, as an example, the course of the drop cycle set up on a grid structure is shown, in which, on the left side is shown the top view onto a grid mesh with a grid window 8, grid crosspieces 9, 9′, 9″, 9″′ and grid nodes 10, 10′, 10″, 10′″, and the right side shows in each case the part of the corresponding grid structure of an inner element 3 in a sectional representation with a section along line A-A referred to the individual grid meshes shown as representative on the left. The suspension containing the organisms (for example, microalgae), which trickles down over the grid structure via introduction organ 2, for example, a spray nozzle, penetrates grid window 8 and in each case, first forms a thin, film-like layer in regions at the grid nodes 10, 10′, 10″, 10″′ and grid crosspieces 9, 9′, 9″, 9′″ below the respective mesh of a grid structure. Due to trailing parts of the suspension, a drop finally begins to appear in the region of grid nodes 10, 10′, 10″, 10″′. This develops into a drop 4 that is subsequently gradually enlarged. Turbulence arises within drop 4 due to the light striking drop 4 and the heating that accompanies it, as well as due to material transport inside the drop, as a consequence of which the microalgae contained in the suspension repeatedly arrive at the drop surface, where they generally stay for a certain period of time and take up energy in the form of light and nutrients (in particular CO₂) from the gaseous environment in the culturing space in a particularly good manner. Drop 4 then grows to a maximum size dependent on its specific surface tension and a constriction is formed on its upper side in the transition region to the grid structure. Finally drop 4 drops off, whereupon it either strikes another grid structure of an inner element 3, 3₁, 3ₙ or moves in the direction of the collecting region 6 at the bottom of culturing space 1. Depending on the configuration of the photobioreactor, this culturing space can also be optionally created such that drop 4 runs down on strips, cords, or chains, or vertical nets 7 disposed under the grid structure.

FIG. 3 relates to the detail X of FIG. 1 in an enlarged spatial representation, according to which appropriate strips, cords or the like as named above are disposed underneath a grid structure. These are, for example, ultrathin strips, by means of which drops 4 are guided down from grid nodes 10, 10′, 10″, 10″′ of the grid structure of an inner element 3, 3₁, 3ₙ to the bottom of culturing space 1, with formation of thin layers on strips 7.

The corresponding strips 7 are preferably at least slightly hydrophilic, so that there is a partial immobilizing of the biomass. The corresponding depositions are then rinsed off from strips 7, preferably in connection with the harvesting of the biomass.

A possible embodiment of the culturing space 1 of a photobioreactor according to the invention is shown once more in a schematic representation in FIG. 4, whereby, in order to provide light to the phototrophic microalgae that are being cultivated, natural sunlight is used, which passes through culturing space 1 of the photobioreactor, whereby culturing space 1 of the photobioreactor shown by way of example is designed with transparent walls for this purpose. As can be recognized from the figure, in the case of this embodiment, the suspension is introduced via a plurality of introduction organs 2. Several horizontal inner elements 3, 3₁, 3ₙ having grid structures are disposed in culturing space 1, each time in cascade fashion, underneath these introduction organs 2. The drop cycle explained according to FIG. 2 takes place several times on them in each case. Underneath these grid cascades are provided channel-type collecting regions 6, by means of which the dripping-down suspension is conducted downward and finally is again conducted to the introduction organs 2 by means of a pumping system 5, which is not shown in detail.

### LIST OF REFERENCE NUMBERS

1 Culturing space

2 Introduction organ

3, 3, 3n (Horizontal) inner element having grid, screen or net structure

4 Drop

5 Pumping system

6 Collecting region

7 (Vertical) inner element (net, cord, strip or chain)

8 Grid window

9′, 9″, 9″′ Grid crosspiece

10′, 10″, 10″′ Grid nodes

1. A method for producing biomass composed of phototrophic or mixotrophic organisms or cells, in which organisms or cells contained in a suspension are cultivated in a photobioreactor with introduction of energy in the form of natural or artificial light and of at least CO₂ as a gaseous nutrient, and after cultivation of the organisms or cells, the biomass is harvested by separation from the suspension, whereby the suspension is circulated during the cultivation of the microorganisms or cells in the photobioreactor by introducing the suspension via at least one introduction organ in an upper region of a culturing space of the photobioreactor, the downward movement of the suspension in the culturing space that occurs due to the effect of gravity is slowed down by at least one suitable inner element disposed in the culturing space for an intensive exposure of the organisms or cells to light entering into the culturing space or produced therein and for exposure to the one or more gaseous nutrients, and the suspension collecting at the bottom of the culturing space is conducted repeatedly to the at least one introduction organ by means of a pumping system, is hereby characterized in that the suspension is conducted to structures of the one or more inner elements of the culturing space, which are formed for this purpose, for the formation of a plurality of drops, whereby the parts of the suspension converted into drops during passage in the culturing space each pass through at least once a drop cycle having the following stages
a. formation of the drop on the structure provided for this in the culturing space,
b. enlargement of the drop up to a maximum size, whereby the organisms or cells contained in the drop are exposed to light and to the one or more gaseous nutrients during the enlargement of the drop and are multiplied,
c. dripping the drop down into a collection region at the bottom of the culturing space or onto another structure of an inner element that produces drops.

2. The method according to claim 1, further characterized in that phototrophic or mixotrophic microalgae are cultivated for the production of the biomass.

3. The method according to claim 1, further characterized in that the suspension is passed through an appropriate formation of the inner element and/or an appropriate control of its introduction via the at least one introduction organ for the formation of predominantly lens-shaped drops.

4. The method according to claim 1, further characterized in that the suspension is passed through an appropriate configuration of the inner element for a mist-like drop formation.

5. The method according to claim 1, further characterized in that drops formed on the corresponding structures of the culturing space are removed from these structures by vibration effect or shock-like vibrations for the harvesting of the biomass, and the suspension with the organisms or cells contained therein, which collects at the bottom of the culturing space, is then conducted to means for separating the organisms or cells from the suspension.

6. The method according to claim 5, further characterized in that the drops are removed from the structures of the culturing space by means of ultrasound acting on these structures.

7. The method according to claim 1, further characterized in that drops formed on the corresponding structures of the culturing space are blown off from these structures for the harvesting of the biomass, and the suspension with the organisms or cells contained therein, which collects at the bottom of the culturing space, is then conducted to means for separating the organisms or cells from the suspension.

8. The method according to claim 1, further characterized in that for harvesting the biomass, a rinsing fluid is introduced into the culturing space by means of the at least one introduction organ, so that the drops formed on the corresponding structures of the culturing space are rinsed off and the liquid mixture of the suspension with the organisms or cells contained therein and the rinsing fluid, which collects at the bottom of the culturing space, is conducted to means for separating the organisms or cells from the suspension.

9. The method according to claim 4, further characterized in that for harvesting the biomass, the mist-like drops are blown out from the culturing space and in this case are conducted to a condenser and the condensed suspension with the organisms or cells contained therein is conducted to means for separating the organisms or cells from the suspension.

10. The method according to claim 5, further characterized in that films of the suspension that absorb the organisms or cells and that remain culturing space and/or on its inner elements are removed by subsequent rinsing with a rinsing fluid.

11. The method according to claim 10, further characterized in that water is used as a rinsing fluid.

12. The method according to claim 10, further characterized in that the nutrient solution is used as a rinsing fluid.

13. A photobioreactor for cultivating phototrophic organisms or cells having a culturing space, through which passes natural light or artificial light produced outside or inside the culturing space and in which at least CO₂ is introduced as a gaseous nutrient, with at least one introduction organ, by means of which the organisms or cells contained in the suspension are introduced in an upper region of the culturing space for exposure to light and to the one or more gaseous nutrients, having at least one inner element disposed in culturing space, by means of which the downward movement of the suspension in culturing space, which occurs due to the effect of gravity, is delayed, and a pumping system, by means of which the suspension collecting at the bottom of culturing space is conducted repeatedly to the at least one introduction organ for providing circulation, is hereby characterized in that at least one inner element with a grid, screen, or net structure extending horizontally is disposed in culturing space underneath the at least one introduction organ, by means of which the suspension containing the organisms or cells, which has been introduced into culturing space, is conducted through the structure for the formation of a plurality of drops, which, after they have formed, increase in size and in each case, after reaching a maximum drop size, are dripped down into a collecting region at the bottom of the culturing space or onto another structure of an inner element that produces drops and is disposed in culturing space.

14. The photobioreactor according to claim 13, further characterized in that several inner elements that extend horizontally in culturing space, each having a grid, screen or net structure, are disposed in a cascade, one underneath the other.

15. The photobioreactor according to claim 13, further characterized in that the grid, sieve or net structure of the at least one horizontal inner element is composed of a hydrophobic material.

16. The photobioreactor according to claim 13, further characterized in that the grid, sieve or net structure of the at least one horizontal inner element is composed of a transparent material.

17. The photobioreactor according to claim 13, further characterized in that the grid, sieve or net structure of the at least one horizontal inner element is composed of a white material.

18. The photobioreactor according to claim 13, further characterized in that the grid, sieve or net structure of the at least one horizontal inner element is composed of a light-conducting material.

19. The photobioreactor according to claim 13, further characterized in that several nets, cords, strips or chains that extend vertically downward from the horizontal grid, sieve or net structure of one of the inner elements or of the last inner element are disposed in culturing space, by means of which drops of the suspension dripping down from the respective horizontal grid, sieve or net structure run downward in the direction of collecting region of culturing space.

20. The photobioreactor according to claim 19, further characterized in that the nets, cords, strips or chains that extend vertically downward are composed of a hydrophilic material.

21. The photobioreactor according to claim 13, further characterized in that it has a unit for generating shock-like vibrations for removing drops that remain on the grid, screen or net structure of the at least one inner element.

22. The photobioreactor according to claim 13, further characterized in that it has an ultrasonic transmitter, the ultrasonic vibrations of which act to remove drops remaining on the grid, screen or net structure of the at least one inner element.

23. The photobioreactor according to claim 13, further characterized in that it has a fan for removing drops remaining on the grid, screen or net structure of the at least one inner element.
